(19) United States
(12) Patent Application Publication
(10) Pub. No.: US 2026/0325280 A1
(43) Pub. Date: Oct. 22, 2026

(54) SPACING AGENT TO PREVENT RECURRENT URINARY TRACT INFECTION

(71) Applicant: Boston Scientific Scimed, Inc., Maple Grove, MN (US)

(72) Inventors: Cristian Parisi, Amherst, MA (US); Kory Patrick Hamel, Minnetonka, MN (US); Niraj Prasad Rauniyar, Plymouth, MN (US); Bilal Chughtai, Glen Head, NY (US)

(73) Assignee: Boston Scientific Scimed, Inc., Maple Grove, MN (US)

(21) Appl. No.: 19/653,723

(22) Filed: Apr. 21, 2026

Related U.S. Application Data
(60) Provisional application No. 63/792,614, filed on Apr. 22, 2025.

(51) Int. Cl. A61F 2/02; A61L 27/26; A61L 27/52; A61L 27/54

(52) U.S. Cl. CPC A61F 2/02; A61L 27/26; A61L 27/52; A61L 27/54; A61F 2250/0003; A61F 2250/0067; A61F 2250/0098; A61L 2400/06

(57) ABSTRACT

Use of a spacing agent in the manufacture of a medicament for preventing recurrent urinary tract infections by increasing urethral-anal distance in a female human is disclosed. The spacing agent may comprise at least one of a hydrogel, a balloon structure, an insertable device or a gel-filled implant. The spacing agent may further include therapeutic agents or imaging contrast agents. The hydrogel may be configured to form upon delivery or be pre-crosslinked prior to delivery, and may comprise either biodegradable or non-biodegradable crosslinked polymer matrices. The balloon structure may be configured to be filled with fluids including gases, solutions, suspensions, or hydrogels. The spacing agent may incorporate radiopaque moieties or contrast media for imaging visibility.

### CROSS-REFERENCE TO RELATED APPLICATIONS

The application claims the benefit of U.S. Provisional Patent Application Ser. No. 63/792,614 filed on Apr. 22, 2025, the disclosure of which is incorporated herein by reference.

### TECHNICAL FIELD

The present disclosure pertains to spacing agents configured to be injected or inserted in a perineal region of a female human body to increase urethral-anal distance

### BACKGROUND

Various risk factors and behavioral modifications have been identified in relation to urinary tract infections. In the context of recurrent urinary tract infections (rUTI), modification of behavioral risk factors may provide only limited effectiveness in preventing recurrence.

Non-modifiable anatomical risk factors for rUTI have also been identified. There remains an unmet need for effective interventions that can address the anatomical risk factors associated with rUTI. A shorter urethral-anal distance represents a significant anatomical factor in rUTI susceptibility.

The present application addresses modification of the urethral-anal distance to reduce this anatomical risk factor.

### SUMMARY

The present disclosure is directed to increasing urethral-anal distance in human female bodies via introduction of a spacing agent into the perineal region.

In an example, a spacing agent is used in a method of preventing recurrent urinary tract infections in a female human, where the method includes injecting or inserting the spacing agent into a perineal region of the female human to increase urethral-anal distance.

Alternatively or additionally to any of the examples above, the spacing agent includes an injectable hydrogel.

Alternatively or additionally to any of the examples above, the spacing agent includes an additional agent selected from at least one of a therapeutic agent or an imaging contrast agent.

Alternatively or additionally to any of the examples above, the therapeutic agent includes an antimicrobial agent.

Alternatively or additionally to any of the examples above, the imaging contrast agent is a radiopaque agent.

Alternatively or additionally to any of the examples above, the therapeutic agent has controlled release properties through a hydrolysable linkage between a crosslinked structure and the therapeutic agent.

Alternatively or additionally to any of the examples above, the injectable hydrogel is configured to form upon delivery or the injectable hydrogel is pre-crosslinked prior to delivery.

Alternatively or additionally to any of the examples above, the injectable hydrogel is configured to exhibit shear-thinning properties during delivery.

Alternatively or additionally to any of the examples above, the injectable hydrogel includes a biodegradable crosslinked polymer matrix or the injectable hydrogel includes a non-biodegradable crosslinked polymer matrix.

Alternatively or additionally to any of the examples above, the injectable hydrogel includes a multi-arm polyethylene glycol polymer functionalized with activated end groups configured to react with a crosslinking agent to form crosslinks.

In an example, a spacing agent is used in the manufacture of a medicament for preventing recurrent urinary tract infections by increasing urethral-anal distance in a female human.

Alternatively or additionally to any of the examples above, the spacing agent includes a hydrogel.

Alternatively or additionally to any of the examples above, the spacing agent includes an additional agent selected from at least one of a therapeutic agent or an imaging contrast agent.

Alternatively or additionally to any of the examples above, the hydrogel is configured to form upon delivery or the hydrogel is pre-crosslinked prior to delivery.

Alternatively or additionally to any of the examples above, the hydrogel is configured to exhibit shear-thinning properties during delivery.

In an example, a method of preventing recurrent urinary tract infections includes introducing a spacing agent into a perineal region of a human female patient to increase a urethral-anal distance.

Alternatively or additionally to any of the examples above, the spacing agent includes at least one of a hydrogel, a fluid filled balloon, an insertable device, or a gel-filled implant.

Alternatively or additionally to any of the examples above, the insertable device is a fluid filled implant.

Alternatively or additionally to any of the examples above, the spacing agent includes an insertable prosthesis.

Alternatively or additionally to any of the examples above, the insertable prosthesis includes a silicone implant.

Alternatively or additionally to any of the examples above, the spacing agent includes an additional agent selected from at least one of a therapeutic agent or an imaging contrast agent.

Alternatively or additionally to any of the examples above, the therapeutic agent is an antimicrobial agent.

Alternatively or additionally to any of the examples above, the imaging contrast agent is a radiopaque agent.

Alternatively or additionally to any of the examples above, the radiopaque agent is mixed with the additional agent or the radiopaque agent is covalently linked to the additional agent.

Alternatively or additionally to any of the examples above, the spacing agent and the additional agent are mixed immediately prior to introduction of the spacing agent or the additional agent is pre-loaded into the spacing agent prior to introduction of the spacing agent into the perineal region.

Alternatively or additionally to any of the examples above, the therapeutic agent is configured for controlled release through degradation of the spacing agent.

Alternatively or additionally to any of the examples above, therapeutic agent is configured for controlled release through degradation of a hydrolysable linkage between a crosslinked structure and the therapeutic agent.

Alternatively or additionally to any of the examples above, the spacing agent includes a biodegradable crosslinked polymer matrix or the spacing agent is non-degradable.

Alternatively or additionally to any of the examples above, the spacing agent is introduced by injection, where the injection includes at least one of transperineal injection, transrectal injection, transurethral injection, or transvaginal injection.

Alternatively or additionally to any of the examples above, the hydrogel is configured to exhibit shear-thinning properties during delivery.

Alternatively or additionally to any of the examples above, the hydrogel includes a multi-arm polyethylene glycol polymer functionalized with activated end groups configured to react with a crosslinking agent to form crosslinks.

Alternatively or additionally to any of the examples above, the spacing agent is a fluid composition, where the fluid composition is configured to form covalent or ionic crosslinks after introduction into the perineal region.

Alternatively or additionally to any of the examples above, introducing the spacing agent includes inserting a balloon into the perineal region of the patient and injecting a fluid filler into the inserted balloon.

Alternatively or additionally to any of the examples above, the fluid is selected from a gas, a solution, a suspension, a shear-thinning hydrogel, or hydrogel precursors that form crosslinked hydrogels within the balloon after injection of the fluid filler.

Alternatively or additionally to any of the examples above, introducing the spacing agent is configured to achieve an increase in urethral-anal distance of at least 5 percent relative to an initial urethral-anal distance.

Alternatively or additionally to any of the examples above, the method includes introducing a 1 milliliter to 50 milliliters volume of the spacing agent into the perineal region.

In an example, a spacing agent is used in a method of preventing recurrent urinary tract infections in a female human, where the method includes injecting the spacing agent into a perineal region of the female human to increase urethral-anal distance.

In an example, a spacing agent is used in the manufacture of a medicament for preventing recurrent urinary tract infections by increasing urethral-anal distance in a female human.

### BRIEF DESCRIPTION OF THE DRAWINGS

FIG. 1A illustrates an injection site for the spacing agent in accordance with an embodiment of the present disclosure.

FIG. 1B illustrates the injection site after introduction of the spacing agent.

### DETAILED DESCRIPTION

The present disclosure is directed to increasing urethral-anal distance in human female bodies via introduction of a spacing agent into the perineal region.

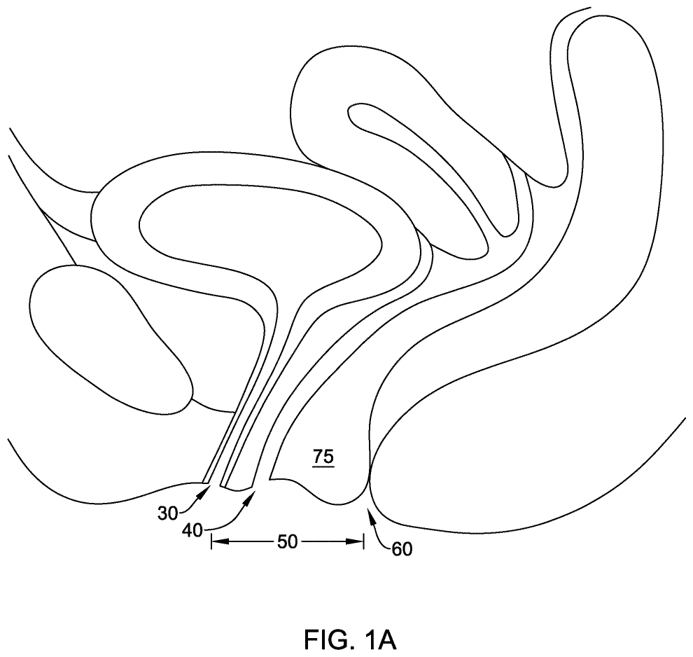

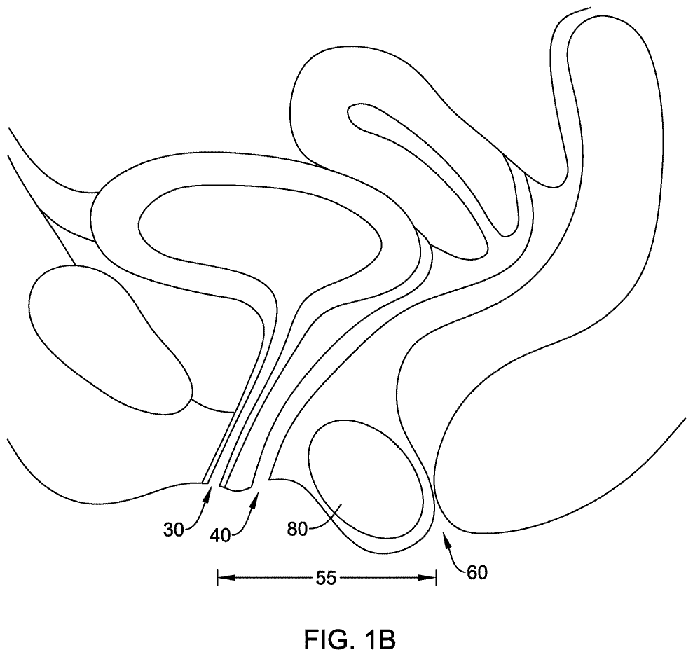

FIG. 1A shows the urethral-anal distance 50 of a patient prior to injection of the spacing agent. The urethral-anal distance 50 extends from the urethra 30 to the anterior rectum 60.

The spacing agent may be introduced into the perineal region through direct injection, balloon-based implantation, a gel-filled implant, or via an insertable device. The perineal region 75 of the patient exists between the posterior vaginal wall 40 and the anterior rectum 60.

In some embodiments, the spacing agent may be injected into muscle tissue, fat tissue, and/or interstitial tissue between the muscle and fat tissues within the perineal region of the patient. The spacing agent can be injected through various approaches including transperineal injection, transrectal injection, transurethral injection, or transvaginal injection.

In some embodiments, the spacing agent is a balloon structure configured to be implanted into a human body at a target location within the perineal region. Once the balloon is implanted, any filler material, precursors, and any additional agents may be introduced into the balloon. The filler materials may include fluids, such as gases, liquids, including solutions and suspensions, and hydrogels, discussed in greater detail below.

After implantation and introduction, the filler material may be released. In some embodiments, the filler material may be released from the balloon over time through diffusion through the balloon wall. In other embodiments, the balloon is formed of a polymeric material configured to degrade through various mechanisms including, but not limited to, hydrolysis by interaction with water in the body, enzymatic degradation by enzymes that are naturally occurring in the body, oxidation by oxidants produced by the body, and/or breakdown using cleavage compositions that are introduced into the body. In embodiments where the filler material contains hydrogel materials with specific crosslinks, degradation can be initiated on demand by contacting the hydrophilic polymer hydrogel compositions with a cleavage composition. The cleavage composition contains a singly reactive molecule that acts to break the crosslinks at specific positions, thereby accelerating the breakdown of the balloon and release of its contents.

In some embodiments, the spacing agent is a silicone gel-filled implant. The silicone gel-filled implant may have a silicone rubber shell formed from polysiloxanes, such as polydimethylsiloxane and polydiphenylsiloxane. The silicone rubber shell is configured to be filled with silicone-based hydrogels and positioned between the posterior vaginal wall and anterior rectum to create an increase in the patient's urethral-anal distance.

In some embodiments, the spacing agent is an insertable device. The device may or may not have filler material. In some embodiments, the insertable device is a homogenous implant or an insertable prosthesis that mechanically create a greater urethral-anal distance. An example of an insertable prosthesis includes, but is not limited to, a silicone implant without any filler material or filler material precursors within. In other embodiments, the insertable device is configured to be filled with a filler material (such as hydrogels or silicone gels discussed above), filler material precursors, and any additional agents.

Prior to injection, a volume for deployment may be created.

The physician may fine-tune the injection volume to achieve the desired anatomical spacing while maintaining patient comfort and proper anatomical function. In some embodiments, the injection volume may range from about 1 milliliter to about 50 milliliters of the spacing agent, for example, ranging anywhere from 1 to 2 to 5 to 10 to 15 to 20 to 30 to 40 to 50 milliliters (in other words, ranging between two of the preceding values).

The injection volume may be selected based on several factors. The minimum volume needed may be determined by the minimum desired increase in urethral-anal distance required to reduce UTI recurrence. The maximum volume may be limited by patient comfort and anatomical constraints. For example, in some embodiments, the volume of the spacing agent should be contained entirely within the perineal region. Additionally, the injection volume should not substantially interfere with surrounding anatomical structures or functions. The injection volume of the spacing agent may be selected to avoid impact on the patient's sexual function, urinary function, and ability to void or sit down, and should not cause closure of any sphincter or orifice.

FIG. 1B shows the urethral-anal distance 55 of the patient after injection of the spacing agent 80. Similar to the urethral-anal distance 50 prior to injection, the urethral-anal distance 55 extends from the urethra 30 to the anterior rectum 60.

Depending on the injection volume and patient characteristics, a post-injection urethral-anal distance 55 may increase by at least 5 percent, at least 10 percent, at least 15 percent, at least 20 percent, at least 25 percent, or at least 50 percent relative to the patient's initial urethral-anal distance 50. Because the initial urethral-anal distance 50 varies significantly between patients, the target increase should be customized based on the individual's anatomy. The maximum increase of the patient's urethral-anal distance 55 is limited primarily, but not exclusively, by patient comfort and safety considerations, with the upper threshold being the point at which the patient experiences pain or discomfort or at which function is adversely impacted. Other factors affecting the achievable increase include, but are not limited to: injection volume; the specific location and technique of injection into the perineal region; the type of spacing agent used and its physical properties; and complications related to sexual function and behavioral modifications (e.g., voiding) that could prevent urinary tract infections (UTIs).

After injection, proper placement of the injection may be confirmed through various imaging modalities including, but not limited to, rectal examination, direct visual inspection, and ultrasound, or X-ray based imaging techniques, such as computerized tomography (CT) or X-ray fluoroscopy. Incorporation of imaging agents, later discussed, may help confirm proper placement and measure the increase in urethral-anal distance.

Spacing agents in accordance with the present disclosure may be a biocompatible composition suited for introduction into the perineal region of the patient to increase the urethral-anal distance of the patient.

Spacing agents in accordance with the present disclosure may include a hydrogel, a balloon, or a gel-filled implant alone or in combination with additional agents, such as a therapeutic agent or an imaging contrast agent.

As used herein, a “hydrogel” is a crosslinked polymer that contains water or can absorb water but does not dissolve when placed in water, although hydrogels may degrade in vivo over time or be degraded by introduction of ex vivo substances.

The hydrogels may be configured to form upon delivery of the hydrogel or may be pre-crosslinked prior to delivery.

In some embodiments, hydrogel precursors may be injected to produce the desired hydrogel upon delivery of the precursors or when otherwise combined. In some embodiments, hydrogel precursors may be injected to produce the desired hydrogel upon delivery or when otherwise combined through in situ crosslinking reactions. The crosslinking process can be initiated by simultaneously injecting precursor solutions with a buffer solution, where the buffer increases pH to accelerate the crosslinking reaction between the components, forming a crosslinked hydrogel within seconds.

An example of such a crosslinked hydrogel is SpaceOAR®, which is based on a multi-arm polyethylene glycol (PEG) polymer functionalized with succinimidyl glutarate as activated end groups which further react with trilysine to form crosslinks. During use, a solution of the multi-arm polymer and the lysine is simultaneously injected with a buffer solution. When mixed, the buffer solution increases the pH and dramatically accelerates the rate of reaction between the multi-arm polymer and the lysine, forming a crosslinked hydrogel within seconds. Another example of such a crosslinked hydrogel is SpaceOAR Vue®, which, like SpaceOAR®, is based on a multi-arm polyethylene glycol (PEG) polymer functionalized with succinimidyl glutarate as activated end groups which further react with trilysine to form crosslinks. In SpaceOAR Vue®, some of the succinimidyl glutarate end groups are functionalized with 2,3,5-triiiodobenzamide groups, providing radiopacity. In other embodiments, systems of these types can be used to form hydrogels ex vivo, after which the hydrogels are broken down into particles and suspended in an aqueous solution to form an injectable pre-formed hydrogel.

In a particular example, a multi-arm PEG-trilysine hydrogel is injected and deposited superficially into the perineal region of cadaver subjects between the posterior vaginal wall and the anterior rectum to achieve an increase in the urethral-anal distance. The pre-injection urethral-anal distance is measured for each subject prior to injection. Post-injection, the mass was surgically removed, and it was found that the hydrogel remains confined within the perineal region as a single, confined mass without infiltration or migration into the surrounding vaginal or rectal tissue. The post-operative urethral-anal distances were measured from photographs of the subjects. The results showed an average increase of 8.6 millimeters in the urethral-anal distance, or an increase of 18%. Detailed results are shown below in Table 1.

TABLE 1
Urethral-Anal Distance Measurements Following
Multi-arm PEG-Trilysine Hydrogel Injection

| | Pre-Injection U-A | Post-Injection U-A | Change in U-A |
|---|---|---|---|
| | Distance (mm) | Distance (mm) | Distance (mm) |
| Subject 1 | 48.0 mm | 57.0 mm | 9.0 mm |
| Subject 2 | 67.5 mm | 73.0 mm | 5.5 mm |
| Subject 3 | 53.0 mm | 58.0 mm | 5.0 mm |
| Subject 4 | 26.0 mm | 41.0 mm | 15.0 mm |
| Average | 48.6 mm | 57.3 mm | 8.6 mm |

The crosslinks formed during delivery may comprise hydrolysable linkers, including carbonate linkers, ester linkers, acid anhydride linkers, imide linkers, ketal linkers, carbamate linkers, organophosphate ester linkers, silane linkers, amide linkers, hydrozonium linkers, acylhydrozone linkers, oxime linkers, and amidohydrozone linkers. The crosslinks may also comprise immolative linkers or reversible covalent linkages that incorporate reactive dimeric linkers dispersed throughout the hydrogels.

In some embodiments, the hydrogel may be formed upon delivery from a lower critical solution temperature (LCST) material, where the LCST refers to the temperature at which the transition from a liquid phase to a hydrogel phase occurs. When the LCST material is injected into a body, the temperature of the LCST material is below the LCST and increases due to heat transfer from the body. Once the temperature of the LCST material reaches the LCST, the transition from the liquid phase to the hydrogel phase takes place. Such LCST materials can be configured or prepared to remain in a liquid phase, with a low viscosity, at an injection temperature below the body temperature (e.g., room temperature or below) and to transform to a gel phase when increased in temperature to body temperature. Examples of such materials include polyoxyethylene-polyoxyproplyene (PEO-PPO) block copolymers, such as Pluronic acid F127 and F108, and N-Isopropyl acrylamide (IPAAm) copolymers.

In some embodiments, hydrogels for use herein are formed from non-degradable crosslinkable materials or injectable particles to form permanent hydrogels.

The non-degradable hydrogels comprise crosslinked networks that contain water or can absorb water but do not dissolve when placed in water. The hydrophilic polymer chains forming these permanent networks may be selected from polyalkylene oxide chains, polyester chains, polyoxazoline chains, polydioxanone chains, polypeptide chains, polyacrylate chains, polyacrylamide chains or combinations thereof. In various embodiments, the polymer chains are crosslinked by multifunctional crosslinkers that contain two or more functional groups (e.g., between two and ten functional groups). The multifunctional crosslinkers have functional groups that irreversibly crosslink with functional groups at ends of the polymer chains.

Examples of suitable materials include but are not limited to polysaccharides, such as hyaluronic acid-based hydrogels, or other hydrophilic polymers, such as but not limited to polyethylene glycol (PEG), modified PEG, polyoxazolines, polypeptides, gelatin, pectin, alginate, cellulose, gellan gum, polyvinylpyrrolidone, poly(2-hydroxyethyl methacrylate), poly(4-hydroxybutyl methacrylate), polyurethanes, polyamides, polyureas, hydrogenated polybutadiene, polybutadiene, polyisobutylene, Kraton styrene block copolymers, and all other derivatives from these polymers.

In some embodiments, hydrophilic polymer hydrogels contain covalent crosslinks between polymer chains, with hydrolysable linkers dispersed throughout. These hydrogels can be degraded in situ by hydrolysis, among other possible mechanisms.

In some embodiments, degradable polymeric materials may be selected from hydrophilic polymer hydrogels that comprise crosslinks between hydrophilic polymer chains within the hydrophilic polymer hydrogel. The hydrophilic polymer chains for use herein are soluble in water and can be cleared from the body, for example, by metabolic processes, clearance through the kidneys and/or other physiological processes.

Degradable polymeric materials may be selected from the following polymers: polyesters including polylactones such as polyvalerolactone and poly(1-lactide-co-ε-caprolactone), polycarbonates such as poly(hexamethylene carbonate) (polyHMC), poly(tetramethylene ether glycol) (polyTHF), polydioxane, poly(lactide co-glycolide), polyethylene glycol, polyvinyl alcohol, water-soluble polyacrylates (e.g. polyacrylic acid), polyoxazolines, polypeptides and proteins. Degradation may occur due to various mechanisms that include hydrolysis by interaction with water in the body, enzymatic degradation by enzymes that are naturally occurring in the body or enzymes that are introduced into the body, oxidation by oxidants produced by the body.

In some embodiments, the crosslinked hydrophilic polymer chains are crosslinked by crosslinks that comprise hydrolysable linkers that degrade upon contact with water. The hydrolysable linkers may be selected from a carbonate linker, an ester linker, an acid anhydride linker, an imide linker, a ketal linker, a carbamate linker, an organophosphate ester linker, a silane linker, an amide linker, a hydrozonium linker, an acylhydrozone linker, an oxime linker and an amidohydrozone linker. The crosslinks may further comprise an activating group that is positioned proximate to the hydrolysable linker in order to tune the rate of hydrolysis of the hydrolysable linker and, in particular embodiments, increase the rate of hydrolysis of the hydrolysable linker.

In some embodiments, the selected hydrogel may be degraded by contact with a cleavage composition that is adapted to break down the crosslinks within the hydrophilic polymer hydrogels.

Contact of the cleavage composition with the hydrophilic polymer hydrogel may include, for example, applying the cleavage composition onto a surface of the hydrogel, injecting the cleavage composition into the hydrogel, and so forth. In some embodiments, the cleavage composition contains a singly reactive molecule that acts to break the crosslinks at the position of the immolative linkers within the crosslinks or at the position of the dimeric linkers within the crosslinks. When degradation of the hydrogel is desired, a needle, catheter or other distribution tool may be advanced to the hydrogel. When the cleavage composition contacts the hydrogel, the immolative linkers or the reversible covalent linkages that incorporate reactive dimeric linkers are cleaved, resulting in degradation of the hydrogel.

The cleavage compositions may further include one or more additional agents, including therapeutic agents or imaging contrast agents, as described below.

In some embodiments, multi-precursor systems can be used to form hydrogels ex vivo, after which the hydrogels are broken down into particles and suspended in an aqueous solution to form an injectable pre-formed hydrogel. The hydrogel particles may be formed from polymeric materials including polysaccharides (e.g., solutions of hyaluronic acid, gelatin, pectin, alginate, cellulose, gellan gum, etc.), polyethylene glycol, polyoxazolines, polypeptides, polyacrylates, polyacrylamides, copolymers of polyethylene glycol, and polyvinyl alcohol. Hydrogel particles may vary widely in size, for example, having an average size ranging from 10 to 2000 microns, or from 50 to 950 microns.

In some embodiments, the spacing agent may be in the form of a shear thinning hydrogel, which is able to temporarily fluidize (the viscosity of the material decreases) under shear stress and recover its original mechanical properties after release of the applied stress. Having shear-thinning properties enables the filler materials to be injected as a low viscosity, flowing material. Such shear thinning hydrogels may be based on natural polymers such as gelatin (e.g., Obsidio™ Conformable Embolic from Boston Scientific, a pre-hydrated bioresorbable mixture of gelatin and layered silicate particles, either with or without tantalum powder for radiocontrast), collagen, hyaluronic acid, alginate, and chitosan. Such hydrogels are generally crosslinked based on physical crosslinking mechanisms such as electrostatic interactions or hydrogen bonding. In some embodiments, the injectable shear-thinning hydrogel filler materials are based on natural polymers such as fibrous proteins or polysaccharides such as hyaluronate, alginate, and chitosan. Such hydrogels are generally crosslinked based on physical crosslinking mechanisms such as electrostatic interactions or hydrogen bonding.

In particular embodiments, the injectable shear-thinning hydrogel filler materials of the present disclosure comprise a fibrous protein, silicate particles and water.

Fibrous proteins are made up of polypeptide chains that are elongated and fibrous in nature or have a sheet-like structure. Fibrous proteins for use in the injectable shear thinning hydrogel filler materials of the present disclosure include animal-derived proteins and non-animal-derived proteins. Fibrous proteins for use in the injectable shear thinning hydrogel filler materials of the present disclosure include keratin, elastin, fibroin, myosin, desmin, fibrin, actin, and collagen, including denatured and hydrolyzed forms thereof such as gelatin, polyarginine, polylysine, and spider silk proteins. Specific fibrous proteins for use herein include bovine collagen, porcine collagen, equine collagen, porcine gelatin (e.g., type-A porcine gelatin, gelatin derived from porcine skin, gelatin derived from porcine bones, and the like), bovine gelatin (e.g., type-B bovine gelatin, gelatin derived from bovine skin, gelatin derived from bovine bones, and the like), equine gelatin, avian-derived gelatin and fish-derived gelatin.

Silicate microparticles for use in the injectable shear-thinning hydrogel filler materials of the present disclosure include natural silicate microparticles and synthetic silicate microparticles. Particular examples of silicate microparticles include natural and synthetic silicate layered clays. Natural silicate layered clays include montmorillonite, saponite, hectorite, kaolinite, palygorskite and sepiolite, among others. Synthetic silicate layered clays include lithium magnesium sodium silicates such as Laponite®-based silicate nanoplatelets (e.g., Laponite® XLG-based silicate nanoplatelets, Laponite® XLS-based silicate nanoplatelets, Laponite® XL21-based silicate nanoplatelets, and Laponite® D-based silicate nanoplatelets), Sumecton® SWN and Lucentite™ SWN, BSC File No.: 24-0209PV01 Atty. Docket No.: 2001.3639100 14 magnesium aluminum silicates such as Sumecton® SA, sodium magnesium silicates such as Optigel® SH and SUPLITE-MP, and fluoromica such as Somasif™ ME100, among others. The silicate microparticles for use in the injectable shear-thinning hydrogel filler materials of the present disclosure include microparticles that have a neutral charge, microparticles that have a net positive charge, and microparticles that have a net negative charge. The net charge of the silicate microparticles may depend upon the pH of the injectable shear-thinning hydrogel filler material. In some embodiments, the silicate microparticles have a net positive charge at the pH of the injectable shear-thinning hydrogel filler material. In some embodiments, the silicate microparticles have a negative positive charge at the pH of the of the injectable shear-thinning hydrogel filler material. In some embodiments, the pH of the of the injectable shear-thinning hydrogel filler material pH between 6 and 11, more typically between 8 and 10.

In some embodiments, the silicate microparticles are plate-shaped. In some embodiments, the silicate microparticles are silicate layered clays characterized by a discotic charge distribution on the surface. In some embodiments, the plate-shaped silicate microparticles comprise a positively charged edge and a negatively charged surface. In some embodiments, the overall charge of the silicate microparticles is negative. In some embodiments, the plate-shaped silicate microparticles are from about 5 nm to about 60 nm in diameter, for example, from about 10 nm to about 40 nm in diameter, from about 10 nm to about 30 nm in diameter, or from about 20 to about 30 nm in diameter. In some embodiments, the plate-shaped silicate microparticles are from about 0.5 nm to about 2 nm in thickness, or about 1 nm in thickness.

In embodiments wherein the spacing agent is a balloon, the balloon structure may be configured to be filled with a fluid. Hydrogels, hydrogel suspensions, and hydrogel precursors that form crosslinked hydrogels, discussed above, may be suitable fluid material for the balloon. Other suitable fluids include solutions, suspensions, and gases.

In some embodiments, the spacing agent may be a balloon structure filled with materials in the form of aqueous liquids including aqueous solutions and aqueous dispersions, particular examples of which include normal saline, phosphate buffered saline, 5% dextrose in water (D5W), contrast media including radiographic contrast media such as iodinated contrast media (e.g., Lipiodol® Guerbet LLC, Princeton, NJ, USA), iohexol (Omnipaque™, GE HealthCare Technologies, Inc., Chicago, IL, USA), iodixanol (Visipaque™, GE HealthCare Technologies, Inc.), iopamidol, ioversol, etc.) and barium sulfate, magnetic resonance imaging (MRI) contrast media including gadolinium-containing contrast media, near-infrared (NIR) contrast media such as those containing indocyanine green, methylene blue, sodium fluorescein, and 5-aminolevulinic acid (5-ALA), and positron emission tomography (PET) contrast media such as those containing [18F]fluorodeoxyglucose (FDG) or [18F]sodium fluoride (Na18F).

In some embodiments, the balloon structure may be filled with a gas or gas mixture. Particular examples may include, but not limited to, helium, air, nitrogen, carbon dioxide, or hydrofluorocarbons.

In some embodiments, the spacing agent may incorporate a therapeutic agent.

In some embodiments, the therapeutic agent may be mixed in situ. In other embodiments, the therapeutic agent may come preloaded into the spacing agent prior to introduction of the spacing agent into the perineal region of the patient.

In some embodiments, the therapeutic agent is an antimicrobial agent suited to treat an outstanding UTI. Incorporation of the antimicrobial agent may offer a treatment regimen that treats both an outstanding UTI and offers prevention of recurrence. Suitable antimicrobial agents may be selected based on the causative organism/pathogen, the spectrum of antimicrobial activity and resistance patterns, the pharmacokinetics of the antimicrobial agent, the side effects and safety profile associated with the antimicrobial agent, duration of treatment, and other patient factors (e.g., pregnancy). Suitable classes antimicrobial agents suited for treatment of a UTI include, but are not limited to, beta-tams, sulfonamides, fluoroquinolones, nitrofurantoin, penicillins, fosfomycin, tetracyclines, and aminoglycosides.

In some embodiments, therapeutic agents may be incorporated into the hydrogel filler materials through hydrolysable linkages between the crosslinked structure and the therapeutic agent to provide controlled release properties. The therapeutic agents may include antithrombotic agents, anticoagulant agents, antiplatelet agents, thrombolytic agents, antiproliferative agents, anti-inflammatory agents, hyperplasia inhibiting agents, anti-restenosis agents, smooth muscle cell inhibitors, antibiotics, antimicrobials, polylysine agents, analgesics, anesthetics, growth factors, growth factor inhibitors, cell adhesion inhibitors, cell adhesion promoters, anti-angiogenic agents, cytotoxic agents, chemotherapeutic agents, checkpoint inhibitors, immune modulatory cytokines, T-cell agonists, STING agonists, antimetabolites, alkylating agents, microtubule inhibitors, hormones, hormone antagonists, monoclonal antibodies, antimitotics, immunosuppressive agents, tyrosine and serine/threonine kinases, proteasome inhibitors, mRNA, matrix metalloproteinase inhibitors, Bcl-2 inhibitors, DNA alkylating agents, spindle poisons, and poly (DP-ribose) polymerase (PARP) inhibitors.

The hydrolysable linkages connecting the therapeutic agents to the crosslinked structure may be selected from a carbonate linkage, an acid anhydride linkage, ester linkage, an imide linkage, a ketal linkage, a carbamate linkage, an organophosphate ester linkage, a silane linkage, an amide linkage, a hydrozonium linkage, an acylhydrozone linkage, an oxime linkage, or an amidohydrozone linkage. The crosslinks may further comprise an activating group positioned adjacent to the hydrolysable linkages, which increases the rate of hydrolysis of the hydrolysable linkages and thereby controls the release rate of the therapeutic agents.

When imaging visibility is desired, the spacing agent may incorporate radiopaque moieties, contrast media, or other imaging agents to enable visualization during and after introduction of the spacing agent into the perineal region. This imaging capability may allow for confirmation of proper placement and assessment of the achieved increase in urethral-anal distance, among other reasons.

In some embodiments, the spacing agent may include radiopaque moieties added to the formulation to provide visibility under X-ray, including X-ray fluoroscopy and/or CT/Cone Beam CT (CBCT) imaging techniques. The radiopaque moieties can include radiopaque atoms such as iodine, barium, bismuth, bromine, strontium, ytterbium, gadolinium, tantalum, titanium, tungsten, platinum, gold, silver, copper, molybdenum, and others. In some embodiments, other radiocontrast agents may be included, for example, particles of tantalum, tungsten, rhenium, niobium, molybdenum, gold, and their alloys, which metallic particles may be spherical or non-spherical. Additional examples of radiocontrast agents include non-ionic radiocontrast agents, such as iohexol, iodixanol, ioversol, iopamidol, ioxilan, or iopromide, ionic radiocontrast agents such as diatrizoate, iothalamate, metrizoate, or ioxaglate, and iodinated oils, including ethiodized poppyseed oil (available as Lipiodol®).

In some embodiments, contrast media other than or in addition to radiocontrast media may be added to the spacing agent. Examples of contrast media for use in conjunction with magnetic resonance imaging (MRI), including contrast agents that contain elements that form paramagnetic ions, such as Gd(III), Mn(II), Fe(III) and compounds (including chelates) containing the same, such as gadolinium ion chelated with diethylenetriaminepentaacetic acid. Examples of contrast media for use in conjunction with ultrasound imaging, including organic and inorganic echogenic particles (i.e., particles that result in an increase in the reflected ultrasonic energy) or organic and inorganic echolucent particles (i.e., particles that result in a decrease in the reflected ultrasonic energy). In some embodiments, the spacing agent may also achieve ultrasound visibility due to the high water content in the formulation of the gel-filled implant. Examples of contrast media or using in connection with near-infrared (NIR) imaging, which can be selected to impart near-infrared fluorescence to the hydrogels of the present disclosure, allowing for deep tissue imaging and device marking, for instance, NIR-sensitive nanoparticles such as gold nanoshells, carbon nanotubes (e.g., nanotubes derivatized with hydroxy or carboxyl groups, for instance, partially oxidized carbon nanotubes), dye-containing nanoparticles, such as dye-doped nanofibers and dye-encapsulating nanoparticles, and semiconductor quantum dots, among others, and NIR-sensitive dyes such as cyanine dyes, squaraines, phthalocyanines, porphyrin derivatives and boron dipyrromethane (BODIPY) analogs, among others, and imageable radioisotopes including 99mTc, 201Th, 51Cr, 67Ga, 68Ga, 111In, 64Cu, 89Zr, 59Fe, 42K, 82Rb, 24Na, 45Ti, 44Sc, 51Cr and 177Lu, among others.

In some embodiments, the spacing agent may include other imaging agents, such as fluorescent dyes and colorants. Examples of fluorescent dyes include fluorescein, indocyanine green, or fluorescent proteins (e.g., green, blue, cyan fluorescent proteins). Other colorants may also be used. Examples of colorants include brilliant blue (e.g., Brilliant Blue FCF, also known as FD&C Blue 1), indigo carmine (also known as FD&C Blue 2), indigo carmine lake, FD&C Blue 1 lake, and methylene blue (also known as methylthioninium chloride), among others.

1. A method of preventing recurrent urinary tract infections, comprising:
introducing a spacing agent into a perineal region of a human female patient to increase a urethral-anal distance.

2. The method of claim 1, wherein the spacing agent comprises at least one of a hydrogel, a fluid filled balloon, an insertable device, or a gel-filled implant.

3. The method of claim 1, wherein the spacing agent comprises an additional agent selected from at least one of a therapeutic agent or an imaging contrast agent.

4. The method of claim 3, wherein the therapeutic agent is an antimicrobial agent.

5. The method of claim 3, wherein the imaging contrast agent is a radiopaque agent.

6. The method of claim 5, wherein the radiopaque agent is mixed with the additional agent or wherein the radiopaque agent is covalently linked to the additional agent.

7. The method of claim 3, wherein the spacing agent and the additional agent are mixed immediately prior to introduction of the spacing agent or wherein the additional agent is pre-loaded into the spacing agent prior to introduction of the spacing agent into the perineal region.

8. The method of claim 3, wherein the therapeutic agent is configured for controlled release through degradation of the spacing agent.

9. The method of claim 3, wherein therapeutic agent is configured for controlled release through degradation of a hydrolysable linkage between a crosslinked structure and the therapeutic agent.

10. The method of claim 1, wherein the spacing agent comprises a biodegradable crosslinked polymer matrix or wherein the spacing agent is non-degradable.

11. The method of claim 1, wherein the spacing agent is introduced by injection, wherein injection comprises at least one of transperineal injection, transrectal injection, transurethral injection, or transvaginal injection.

12. The method of claim 2, wherein the hydrogel is configured to exhibit shear-thinning properties during delivery.

13. The method of claim 2, wherein the hydrogel comprises a multi-arm polyethylene glycol polymer functionalized with activated end groups configured to react with a crosslinking agent to form crosslinks.

14. The method of claim 1, wherein the spacing agent is a fluid composition, wherein the fluid composition is configured to form covalent or ionic crosslinks after introduction into the perineal region.

15. The method of claim 1, wherein introducing the spacing agent further comprises inserting a balloon into the perineal region of the patient and injecting a fluid filler into the inserted balloon.

16. The method of claim 15, wherein the fluid filler is selected from a gas, a solution, a suspension, a shear-thinning hydrogel, or hydrogel precursors that form crosslinked hydrogels within the balloon after injection of the fluid filler.

17. The method of claim 1, wherein introducing the spacing agent is configured to achieve an increase in urethral-anal distance of at least 5 percent relative to an initial urethral-anal distance.

18. The method of claim 1, further comprising introducing a 1 milliliter to 50 milliliters volume of the spacing agent into the perineal region.

19. A spacing agent for use in a method of preventing recurrent urinary tract infections in a female human, the method comprising injecting the spacing agent into a perineal region of the female human to increase urethral-anal distance.

20. Use of a spacing agent in the manufacture of a medicament for preventing recurrent urinary tract infections by increasing urethral-anal distance in a female human.
